(19) United States
(12) Patent Application Publication
(10) Pub. No.: US 2013/0071422 A1
(43) Pub. Date: Mar. 21, 2013

(54) ADJUVANTED VACCINES FOR SEROGROUP B MENINGOCOCCUS

(71) Applicants: Michele Pallaoro, Siena (IT); Derek O'Hagan, Winchester, MA (US); Rino Rappuoli, Castelnuovo Berardenga (IT)

(72) Inventors: Michele Pallaoro, Siena (IT); Derek O'Hagan, Winchester, MA (US); Rino Rappuoli, Castelnuovo Berardenga (IT)

(21) Appl. No.: 13/634,852

(22) PCT Filed: Mar. 18, 2011
(86) PCT No.: PCT/IB11/51148

§ 371 Date: Sep. 13, 2012

Related U.S. Application Data
(60) Provisional application No. 61/315,336, filed on Mar. 18, 2010.
(60) Provisional application No. 61/317,572, filed on Mar. 25, 2010.

(51) Int. Cl. A61K 39/095; A61K 39/13; A61K 39/29; A61K 39/39; A61K 39/02

(52) U.S. Cl. CPC A61K 39/095; A61K 39/39; A61K 39/099; A61K 39/292; A61K 39/29; A61K 39/13
USPC 424/189.1; 424/190.1; 424/186.1; 424/250.1

(57) ABSTRACT

An immunogenic composition comprises (i) an immuno stimulatory oligonucleotide and a polycationic polymer, wherein the oligonucleotide and the polymer ideally associate with each other to form a complex, and (ii) a meningococcal serogroup B antigen. In most embodiments, the composition does not include an aluminium salt and does not include an oil-in-water emulsion.

This application claims the benefit of U.S. provisional patent applications 61/315,336, filed 18 Mar. 2010, and 61/317,572, filed 25 Mar. 2010, the complete contents of both of which are incorporated herein by reference for all purposes.

### TECHNICAL FIELD

This invention is in the field of meningococcal vaccines.

### BACKGROUND ART

Various vaccines against serogroup B of Neisseria meningitidis (“MenB”) are currently being investigated. Some vaccines are based on outer membrane vesicles (OMVs), such as the Novartis Vaccines MENZB™ product, the Finlay Institute VA-MENGOC-BCT™ product, and the Norwegian Institute of Public Health MENBVAC™ product. Others are based on recombinant proteins, such as the “universal vaccine for serogroup B meningococcus” reported by Novartis Vaccines in ref 1.

It is an object of the invention to provide modified and improved vaccines against MenB and, in particular, adjuvanted vaccines.

### DISCLOSURE OF THE INVENTION

The invention provides an immunogenic composition comprising (i) a meningococcal serogroup B antigen and (ii) an adjuvant comprising an immunostimulatory oligonucleotide and a polycationic polymer; wherein (i) the immunogenic composition does not include an aluminium salt; (ii) the immunogenic composition does not include an oil-in-water emulsion; (iii) the meningococcal serogroup B antigen does not include a polypeptide comprising an amino acid sequence selected from SEQ ID NOs 13, 14, 15, 16, 17, 18, 19, 20, 21 or 22; and (iv) the immunogenic composition does not include a fHBP antigen.

The immunostimulatory oligonucleotide and polycationic polymer preferably associate with each other. They can form an oligonucleotide/polymer complex.

The invention also provides an immunogenic composition comprising (i) a meningococcal serogroup B antigen; (ii) an adjuvant comprising an immunostimulatory oligonucleotide and a polycationic polymer and; (iii) one or more further antigens selected from a pneumococcal antigen, a diphtheria toxoid, tetanus toxoid, a pertussis antigen, HBsAg, a HAV antigen, a Hib antigen, and/or IPV. The immunogenic composition can also include an aluminium salt and/or an oil-in-water emulsion.

The invention also provides an immunogenic composition comprising (i) a purified meningococcal lipooligosaccharide; and (ii) an adjuvant comprising an immunostimulatory oligonucleotide and a polycationic polymer. The immunogenic composition can also include an aluminium salt and/or an oil-in-water emulsion.

The invention also provides an immunogenic composition comprising (i) an 5-valent antigen component consisting of a MenB antigen, a conjugated capsular saccharide from serogroup A N. meningitidis, a conjugated capsular saccharide from serogroup C N. meningitidis, a conjugated capsular saccharide from serogroup W135 N. meningitidis, a conjugated capsular saccharide from serogroup Y N. meningitidis; and (ii) an adjuvant comprising an immunostimulatory oligonucleotide and a polycationic polymer, provided that the immunogenic composition does not include an aluminium salt and does not include an oil-in-water emulsion.

In one embodiment of the invention, the MenB antigen can be adsorbed to a complex formed by the oligonucleotide and polymer in the adjuvant. Alternatively, the MenB antigen is not adsorbed to the oligonucleotide/polymer complex in the adjuvant.

The invention also provides a process for preparing an immunogenic composition of the invention, comprising a step of mixing (i) an adjuvant comprising a complex of an immunostimulatory oligonucleotide and a polycationic polymer and (ii) a meningococcal serogroup B (“MenB”) antigen, provided that the MenB antigen does not include a polypeptide comprising an amino acid sequence selected from SEQ ID NOs 13, 14, 15, 16, 17, 18, 19, 20, 21 or 22 and does not include a fHBP antigen. In alternative methods, the MenB antigen and adjuvant comprising an immunostimulatory oligonucleotide and polycationic polymer are mixed before the complex has formed. For example, the MenB antigen can be mixed with the oligonucleotide, and then the polymer is added; or the MenB antigen can be mixed with the polymer, and then the oligonucleotide is added. The complex may form after the oligonucleotide and the polymer meet.

The MenB antigen, oligonucleotide and polymer may be mixed in any order.

The invention also provides a kit comprising: (i) a first container that contains an immunostimulatory oligonucleotide and a polycationic polymer and (ii) a second container that contains a MenB antigen provided that the MenB antigen does not include a polypeptide comprising an amino acid sequence selected from SEQ ID NOs 13, 14, 15, 16, 17, 18, 19, 20, 21 or 22 and does not include a fHBP antigen Neither the first container nor the second container in the kit includes an aluminium salt or an oil-in-water emulsion.

The invention also provides a kit comprising (i) a first container that contains an immunostimulatory oligonucleotide and a polycationic polymer and (ii) a second container that contains a purified meningococcal lipooligosaccharide.

The invention also provides a kit comprising which comprises (i) a first container that contains an immunostimulatory oligonucleotide and a polycationic polymer and (ii) a second container that contains a meningococcal serogroup B antigen and (iii) a container that contains one or more further antigens selected from a pneumococcal antigen, diphtheria toxoid, tetanus toxoid, a pertussis antigen, HBsAg, a HAV antigen, Hib antigen, and/or IPV. The container mentioned in part (iii) can be the first container, the second container, or a third container.

The contents of the containers in these kits can be combined (e.g. at the point of use) to form an immunogenic composition of the invention. These kits may include a further container that contains an immunogen and/or a further adjuvant.

In some embodiments, the only adjuvant in a composition or kit is the adjuvant comprising an immunostimulatory oligonucleotide and a polycationic polymer.

### Serogroup B Meningococcus Immunogens

Immunogenic compositions of the invention are useful for eliciting an immune response against serogroup B meningococcus (“MenB”). Suitable immunogens for eliciting anti-MenB responses include polypeptide antigens, lipooligosaccharide and/or membrane vesicles. Further details of useful serogroup B antigens are given below.

### Meningococcal Polypeptide Antigens

An immunogenic composition of the invention may include one or more meningococcal polypeptide antigen(s). For instance, a composition may include a polypeptide antigen selected from the group consisting of: 287, NadA, NspA, HmbR, NhhA, App and/or Omp85. These antigens will usefully be present as purified polypeptides e.g. recombinant polypeptides. The antigen will preferably elicit bactericidal anti-meningococcal antibodies after administration to a subject.

An immunogenic composition of the invention may include a 287 antigen. The 287 antigen was included in the published genome sequence for meningococcal serogroup B strain MC58 [2] as gene NMB2132 (GenBank accession number GI:7227388; SEQ ID NO: 3 herein). The sequences of 287 antigen from many strains have been published since then. For example, allelic forms of 287 can be seen in FIGS. 5 and 15 of reference 3, and in example 13 and FIG. 21 of reference 4 (SEQ IDs 3179 to 3184 therein). Various immunogenic fragments of the 287 antigen have also been reported. Preferred 287 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) comprising a fragment of at least ‘n’ consecutive amino acids of SEQ ID NO: 3, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 3. The most useful 287 antigens of the invention can elicit antibodies which, after administration to a subject, can bind to a meningococcal polypeptide consisting of amino acid sequence SEQ ID NO: 3. Advantageous 287 antigens for use with the invention can elicit bactericidal anti-meningococcal antibodies after administration to a subject.

An immunogenic composition of the invention composition of the invention may include a NadA antigen. The NadA antigen was included in the published genome sequence for meningococcal serogroup B strain MC58 [2] as gene NMB 1994 (GenBank accession number GI:7227256; SEQ ID NO: 4 herein). The sequences of NadA antigen from many strains have been published since then, and the protein's activity as a Neisserial adhesin has been well documented. Various immunogenic fragments of NadA have also been reported. Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘n’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 4. SEQ ID NO: 6 is one such fragment. The most useful NadA antigens of the invention can elicit antibodies which, after administration to a subject, can bind to a meningococcal polypeptide consisting of amino acid sequence SEQ ID NO: 4. Advantageous NadA antigens for use with the invention can elicit bactericidal anti-meningococcal antibodies after administration to a subject.

An immunogenic composition of the invention may include a NspA antigen. The NspA antigen was included in the published genome sequence for meningococcal serogroup B strain MC58 [2] as gene NMB0663 (GenBank accession number GI:7225888; SEQ ID NO: 5 herein). The antigen was previously known from references 5 & 6. The sequences of NspA antigen from many strains have been published since then. Various immunogenic fragments of NspA have also been reported. Preferred NspA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 5. The most useful NspA antigens of the invention can elicit antibodies which, after administration to a subject, can bind to a meningococcal polypeptide consisting of amino acid sequence SEQ ID NO: 5. Advantageous NspA antigens for use with the invention can elicit bactericidal anti-meningococcal antibodies after administration to a subject.

An immunogenic composition of the invention may include a meningococcal HmbR antigen. The full-length HmbR sequence was included in the published genome sequence for meningococcal serogroup B strain MC58 [2] as gene NMB1668 (SEQ ID NO: 12 herein). The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i % sequence identity to SEQ ID NO: 12, where the value of i is 50, 60, 70, 80, 90, 95, 99 or more. In other embodiments a HmbR sequence used according to the invention may comprise a fragment of at least j consecutive amino acids from SEQ ID NO: 12, where the value of j is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i % sequence identity to SEQ ID NO: 12 and/or (ii) comprising a fragment of at least j consecutive amino acids from SEQ ID NO: 12. Preferred fragments of j amino acids comprise an epitope from SEQ ID NO: 12. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 7. The most useful HmbR antigens of the invention can elicit antibodies which, after administration to a subject, can bind to a meningococcal polypeptide consisting of amino acid sequence SEQ ID NO: 12. Advantageous HmbR antigens for use with the invention can elicit bactericidal anti-meningococcal antibodies after administration to a subject.

An immunogenic composition of the invention may include a NhhA antigen. The NhhA antigen was included in the published genome sequence for meningococcal serogroup B strain MC58 [2] as gene NMB0992 (GenBank accession number GI:7226232; SEQ ID NO: 6 herein). The sequences of NhhA antigen from many strains have been published since e.g. refs 3 & 8, and various immunogenic fragments of NhhA have been reported. It is also known as Hsf. Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) comprising a fragment of at least ‘n’ consecutive amino acids of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 6. The most useful NhhA antigens of the invention can elicit antibodies which, after administration to a subject, can bind to a meningococcal polypeptide consisting of amino acid sequence SEQ ID NO: 6. Advantageous NhhA antigens for use with the invention can elicit bactericidal anti-meningococcal antibodies after administration to a subject.

An immunogenic composition of the invention may include an App antigen. The App antigen was included in the published genome sequence for meningococcal serogroup B strain MC58 [2] as gene NMB 1985 (GenBank accession number GI:7227246; SEQ ID NO: 7 herein). The sequences of App antigen from many strains have been published since then. Various immunogenic fragments of App have also been reported. Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) comprising a fragment of at least ‘n’ consecutive amino acids of SEQ ID NO: 7, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 7. The most useful App antigens of the invention can elicit antibodies which, after administration to a subject, can bind to a meningococcal polypeptide consisting of amino acid sequence SEQ ID NO: 7. Advantageous App antigens for use with the invention can elicit bactericidal anti-meningococcal antibodies after administration to a subject.

An immunogenic composition of the invention may include an Omp85 antigen. The Omp85 antigen was included in the published genome sequence for meningococcal serogroup B strain MC58 [2] as gene NMB0182 (GenBank accession number GI:7225401; SEQ ID NO: 8 herein). The sequences of Omp85 antigen from many strains have been published since then. Further information on Omp85 can be found in references 9 and 10. Various immunogenic fragments of Omp85 have also been reported. Preferred Omp85 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 8. The most useful Omp85 antigens of the invention can elicit antibodies which, after administration to a subject, can bind to a meningococcal polypeptide consisting of amino acid sequence SEQ ID NO: 8. Advantageous Omp85 antigens for use with the invention can elicit bactericidal anti-meningococcal antibodies after administration to a subject.

Compositions of the invention do not include meningococcal factor H binding protein (fHBP) antigen. A fHBP antigen is a polypeptide comprising an amino acid sequence, (i) having at least 80% sequence identity to any one of SEQ ID NOs: 9, 10, or 11 and/or (ii) consisting of a fragment of at least 7 contiguous amino acids from SEQ ID NOs: 9, 10 or 11. In some embodiments the compositions do not include a protein which can bind to factor H (e.g. human factor H) in an assay as described in references 11 and 12.

Fragments preferably comprise an epitope from the respective SEQ ID NO: sequence. Other useful fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of the respective SEQ ID NO: while retaining at least one epitope thereof.

In some embodiments polypeptide(s) are lipidated e.g. at a N-terminus cysteine. For lipidated polypeptide(s), lipids attached to cysteines will usually include palmitoyl residues e.g. as tripalmitoyl-S-glyceryl-cysteine (Pam3Cys), dipalmitoyl-S-glyceryl cysteine (Pam2Cys), N-acetyl (dipalmitoyl-S-glyceryl cysteine), etc.

### Meningococcal Lipooligosaccharide

An immunogenic composition may include one or more meningococcal lipooligosaccharide (LOS) antigen(s). Meningococcal LOS is a glucosamine-based phospholipid that is found in the outer monolayer of the outer membrane of the bacterium. It includes a lipid A portion and a core oligosaccharide region, with the lipid A portion acting as a hydrophobic anchor in the membrane. Heterogeneity within the oligosaccharide core generates structural and antigenic diversity among different meningococcal strains, which has been used to subdivide the strains into 12 immunotypes (L1 to L12). The invention may use LOS from any immunotype e.g. from L1, L2, L3, L4, L5, L6, L7 and/or L8.

The L2 and L3 α-chains naturally include lacto-N-neotetraose (LNnT). Where the invention uses LOS from a L2 or L3 immunotype this LNnT may be absent. This absence can be achieved conveniently by using mutant strains that are engineered to disrupt their ability to synthesise the LNnT tetrasaccharide within the α-chain. It is known to achieve this goal by knockout of the enzymes that are responsible for the relevant biosynthetic additions [13,43]. For instance, knockout of the LgtB enzyme prevents addition of the terminal galactose of LNnT, as well as preventing downstream addition of the α-chain's terminal sialic acid. Knockout of the LgtA enzyme prevents addition of the N-acetyl-glucosamine of LNnT, and also the downstream additions. LgtA knockout may be accompanied by LgtC knockout. Similarly, knockout of the LgtE and/or GalE enzyme prevents addition of internal galactose, and knockout of LgtF prevents addition of glucose to the Hep^{I} residue. Any of these knockouts can be used, singly or in combination, to disrupt the LNnT tetrasaccharide in a L2, L3, L4, L7 or L9 immunotype strain. Knockout of at least LgtB is preferred, as this provides a LOS that retains useful immunogenicity while removing the LNnT epitope.

In addition to, or in place of, mutations to disrupt the LNnT epitope, a knockout of the galE gene also provides a useful modified LOS, and a lipid A fatty transferase gene may similarly be knocked out [14]. At least one primary O-linked fatty acid may be removed from LOS [15]. LOS having a reduced number of secondary acyl chains per LOS molecule can also be used [16]. The LOS will typically include at least the GlcNAc-Hep₂phosphoethanolamine-KDO₂-Lipid A structure [17]. The LOS may include a GlcNAcβ1-3Galβ1-4Glc trisaccharide while lacking the LNnT tetrasaccharide.

LOS may be included in various forms. It may be used in purified form on its own. It may be conjugated to a carrier protein. When LOS is conjugated, conjugation may be via a lipid A portion in the LOS or by any other suitable moiety e.g. its KDO residues. If the lipid A moiety of LOS is absent then such alternative linking is required. Conjugation techniques for LOS are known from e.g. references 15, 17, 18, 19, etc. Useful carrier proteins for these conjugates include e.g. bacterial toxins, such as diphtheria or tetanus toxins, or toxoids or mutants thereof.

The LOS may be from a strain (e.g. a genetically-engineered meningococcal strain) which has a fixed (i.e. not phase variable) LOS immunotype as described in reference 20. For example, L2 and L3 LOS immunotypes may be fixed. Such strains may have a rate of switching between immunotypes that is reduced by more than 2-fold (even >50_fold) relative to the original wild-type strain. Reference 20 discloses how this result can be achieved by modification of the IgtA and/or IgtG gene products.

LOS may be O-acetylated on a GlcNac residue attached to its Heptose II residue e.g. for L3 [21].

An immunogenic composition of the invention can include more than one type of LOS e.g. LOS from meningococcal immunotypes L2 and L3. For example, the LOS combinations disclosed in reference 22 may be used.

A LOS antigen can preferably elicit bactericidal anti-meningococcal antibodies after administration to a subject.

### Membrane Vesicles

An immunogenic composition of the invention may include meningococcal outer membrane vesicles. These include any proteoliposomic vesicle obtained by disruption of or blebbling from a meningococcal outer membrane to form vesicles therefrom that include protein components of the outer membrane. Thus the term includes OMVs (sometimes referred to as ‘blebs’), microvesicles (MVs [23]) and ‘native OMVs’ (‘NOMVs’ [24]).

MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 25 & 26 describe Neisseria with high MV production.

OMVs are prepared artificially from bacteria, and may be prepared using detergent treatment (e.g. with deoxycholate), or by non-detergent means (e.g. see reference 27). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [28 & 29] being preferred for treating Neisseria) at a pH sufficiently high not to precipitate the detergent [30]. Other techniques may be performed substantially in the absence of detergent [27] using techniques such as sonication, homogenisation, microfluidisation, cavitation, osmotic shock, grinding, French press, blending, etc. Methods using no or low detergent can retain useful antigens such as NspA [27]. Thus a method may use an OMV extraction buffer with about 0.5% deoxycholate or lower e.g. about 0.2%, about 0.1%, <0.05% or zero.

A useful process for OMV preparation is described in reference 31 and involves ultrafiltration on crude OMVs, rather than instead of high speed centrifugation. The process may involve a step of ultracentrifugation after the ultrafiltration takes place.

Vesicles for use with the invention can be prepared from any meningococcal strain. The vesicles will usually be from a serogroup B strain, but it is possible to prepare them from serogroups other than B (e.g. reference 30 discloses a process for serogroup A), such as A, C, W135 or Y. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. These lineages have been defined by multilocus enzyme electrophoresis (MLEE), but multilocus sequence typing (MLST) has also been used to classify meningococci [ref. 32] e.g. the ET-37 complex is the ST-11 complex by MLST, the ET-5 complex is ST-32 (ET-5), lineage 3 is ST-41/44, etc. Vesicles can be prepared from strains having one of the following subtypes: P1.2; P1.2,5; P1.4; P1.5; P1.5,2; P1.5,c; P1.5c,10; P1.7,16; P1.7,16b; P1.7h,4; P1.9; P1.15; P1.9,15; P1.12,13; P1.13; P1.14; P1.21,16; P1.22,14.

Vesicles used with the invention may be prepared from wild-type meningococcal strains or from mutant meningococcal strains. For instance, reference 33 discloses preparations of vesicles obtained from N. meningitidis with a modified fur gene. Reference 41 teaches that nspA expression should be up-regulated with concomitant porA and cps knockout. Further knockout mutants of N. meningitidis for OMV production are disclosed in references 41 to 43. Reference 34 discloses vesicles in which fHBP is upregulated. Reference 35 discloses the construction of vesicles from strains modified to express six different PorA subtypes. Mutant Neisseria with low endotoxin levels, achieved by knockout of enzymes involved in LPS biosynthesis, may also be used [36,37]. These or others mutants can all be used with the invention.

Thus a strain used with the invention may in some embodiments express more than one PorA subtype. 6-valent and 9-valent PorA strains have previously been constructed. The strain may express 2, 3, 4, 5, 6, 7, 8 or 9 of PorA subtypes: P1.7,16; P1.5-1,2-2; P1,19,15-1; P1.5-2,10; P1.12-1,13; P1.7-2,4; P1.22,14; P1.7-1,1 and/or P1.18-1,3,6. In other embodiments a strain may have been down-regulated for PorA expression e.g. in which the amount of PorA has been reduced by at least 20% (e.g. ≧30%, ≧40%, ≧50%, ≧60%, ≧70%, ≧80%, ≧90%, ≧95%, etc.), or even knocked out, relative to wild-type levels (e.g. relative to strain H44/76, as disclosed in reference 44).

In some embodiments a strain may hyper-express (relative to the corresponding wild-type strain) certain proteins. For instance, strains may hyper-express NspA, protein 287 [38], fHBP [34], TbpA and/or TbpB [39], Cu,Zn-superoxide dismutase [39], HmbR, etc.

In some embodiments a strain may include one or more of the knockout and/or hyper-expression mutations disclosed in references 40 to 43. Preferred genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB [40]; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB [41]; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB [42]; and (d) CtrA, CtrB, CtrD, FrpB, OpA, OpC, PilC, PorB, SiaD, SynA, SynB, and/or SynC [43].

Where a mutant strain is used, in some embodiments it may have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no a chain.

If LOS is present in a vesicle it is possible to treat the vesicle so as to link its LOS and protein components (“intra-bleb” conjugation [43]).

The invention may be used with mixtures of vesicles from different strains. For instance, reference 44 discloses vaccine comprising multivalent meningococcal vesicle compositions, comprising a first vesicle derived from a meningococcal strain with a serosubtype prevalent in a country of use, and a second vesicle derived from a strain that need not have a serosubtype present in a country of use. Reference 45 also discloses useful combinations of different vesicles. A combination of vesicles from strains in each of the L2 and L3 immunotypes may be used in some embodiments.

In some embodiments, the immunogenic composition does not contain MenB OMV.

Immunogenic compositions of the invention can be administered to animals to induce an immune response. The invention can be used for treating or protecting against a wide range of diseases.

### The Immunostimulatory Oligonucleotide and the Polycationic Polymer

The invention uses an immunostimulatory oligonucleotide and a polycationic polymer. These are ideally associated with each other to form a particulate complex, which usefully is a TLR9 agonist.

Immunostimulatory oligonucleotides are known as useful adjuvants. They often contain a CpG motif (a dinucleotide sequence containing an unmethylated cytosine linked to a guanosine) and their adjuvant effect is discussed in refs. 46-51. Oligonucleotides containing TpG motifs, palindromic sequences, multiple consecutive thymidine nucleotides (e.g. TTTT), multiple consecutive cytosine nucleotides (e.g. CCCC) or poly(dG) sequences are also known immunostimulants, as are double-stranded RNAs. Although any of these various immunostimulatory oligonucleotides can be used with the invention, it is preferred to use an oligodeoxynucleotide containing deoxyinosine and/or deoxyuridine [52], and ideally an oligodeoxynucleotide containing deoxyinosine and deoxycytosine. Inosine-containing oligodeoxynucleotides may include a CpI motif (a dinucleotide sequence containing a cytosine linked to an inosine). The oligodeoxynucleotide may include more than one (e.g. 2, 3, 4, 5, 6 or more) CpI motif, and these may be directly repeated (e.g. comprising the sequence (CI)ₓ, where x is 2, 3, 4, 5, 6 or more) or separated from each other (e.g. comprising the sequence (CIN)ₓ, where x is 2, 3, 4, 5, 6 or more, and where each N independently represents one or more nucleotides). Cytosine residues are ideally unmethylated.

The oligonucleotides will typically have between 10 and 100 nucleotides e.g. 15-50 nucleotides, 20-30 nucleotides, or 25-28 nucleotides. It will typically be single-stranded.

The oligonucleotide can include exclusively natural nucleotides, exclusively non-natural nucleotides, or a mix of both. For instance, it may include one or more phosphorothioate linkage(s), and/or one or more nucleotides may have a 2′-O-methyl modification.

A preferred oligonucleotide for use with the invention is a single-stranded deoxynucleotide comprising the 26-mer sequence 5′-(IC)₁₃-3′ (SEQ ID NO: 1). This oligodeoxynucleotide forms stable complexes with polycationic polymers to give a good adjuvant.

The polycationic polymer is ideally a polycationic peptide, such as a cationic antimicrobial peptide. The polymer may include one or more leucine amino acid residue(s) and/or one or more lysine amino acid residue(s). The polymer may include one or more arginine amino acid residue(s). It may include at least one direct repeat of one of these amino acids e.g. one or more Leu-Leu dipeptide sequence(s), one or more Lys-Lys dipeptide sequence(s), or one or more Arg-Arg dipeptide sequence(s). It may include at least one (and preferably multiple e.g. 2 or 3) Lys-Leu dipeptide sequence(s) and/or at least one (and preferably multiple e.g. 2 or 3) Lys-Leu-Lys tripeptide sequence(s).

The peptide may comprise a sequence R₁—XZXZₓXZX—R₂, wherein: x is 3, 4, 5, 6 or 7; each X is independently a positively-charged natural and/or non-natural amino acid residue; each Z is independently an amino acid residue L, V, I, F or W; and R₁ and R₂ are independently selected from the group consisting of —H, —NH₂, —COCH₃, or —COH. In some embodiments X—R₂ may be an amide, ester or thioester of the peptide's C-terminal amino acid residue. See also reference 53.

A polycationic peptide will typically have between 5 and 50 amino acids e.g. 6-20 amino acids, 7-15 amino acids, or 9-12 amino acids.

A peptide can include exclusively natural amino acids, exclusively non-natural amino acids, or a mix of both. It may include L-amino acids and/or D-amino acids. L-amino acids are typical.

A peptide can have a natural N-terminus (NH₂—) or a modified N-terminus e.g. a hydroxyl, acetyl, etc. A peptide can have a natural C-terminus (—COOH) or a modified C-terminus e.g. a hydroxyl, an acetyl, etc. Such modifications can improve the peptide's stability.

A preferred peptide for use with the invention is the 11-mer KLKLLLLLKLK (SEQ ID NO: 2; ref. 54), with all L-amino acids. The N-terminus may be deaminated and the C-terminus may be hydroxylated. A preferred peptide is H-KLKL₅KLK-OH, with all L-amino acids. This oligopeptide is a known antimicrobial [55], neutrophil activator [56] and adjuvant [57] and forms stable complexes with immunostimulatory oligonucleotides to give a good adjuvant.

The most preferred mixture of immunostimulatory oligonucleotide and polycationic polymer is the TLR9 agonist known as IC31™ [58-60], which is an adsorptive complex of oligodeoxynucleotide SEQ ID NO: 1 and polycationic oligopeptide SEQ ID NO: 2.

The oligonucleotide and oligopeptide can be mixed together at various ratios, but they will generally be mixed with the peptide at a molar excess. The molar excess may be at least 5:1 e.g. 10:1, 15:1, 20:1, 25:1, 30; 1, 35:1, 40:1 etc. A molar ratio of about 25:1 is ideal [61,62]. Mixing at this excess ratio can result in formation of insoluble particulate complexes between oligonucleotide and oligopeptide. Where the MenB antigen is purified LOS, the complexes can be combined with an aluminium salt as described herein.

The oligonucleotide and oligopeptide will typically be mixed under aqueous conditions e.g. a solution of the oligonucleotide can be mixed with a solution of the oligopeptide with a desired ratio. The two solutions may be prepared by dissolving dried (e.g. lyophilised) materials in water or buffer to form stock solutions that can then be mixed.

The complexes can be analysed using the methods disclosed in reference 63. Complexes with an average diameter in the range 1 μm-20 μm are typical.

Poly-arginine and CpG oligodeoxynucleotides similarly form complexes [64].

The complexes can be maintained in aqueous suspension e.g. in water or in buffer. Typical buffers for use with the complexes are phosphate buffers (e.g. phosphate-buffered saline), Tris buffers, Tris/sorbitol buffers, borate buffers, succinate buffers, citrate buffers, histidine buffers, etc. As an alternative, complexes may sometimes be lyophilised.

Complexes in aqueous suspension can be centrifuged to separate them from bulk medium (e.g. by aspiration, decanting, etc.). These complexes can then be re-suspended in an alternative medium if desired.

### Aluminium Salts

Most embodiments of the invention do not include an aluminium salt. Some embodiments permit the use of aluminium salts, however; for example, where the immunogenic composition comprises a purified MenB LOS or where the composition includes one or more further antigens selected from pneumococcal saccharide antigen, diphtheria toxoid, tetanus toxoid, pertussis antigen, HBsAg, HAV antigen, Hib antigen and IPV. Aluminium salts include the adjuvants known individually as aluminium hydroxide and aluminium phosphate. These names are conventional, but are used for convenience only, as neither is a precise description of the actual chemical compound which is present [e.g. see chapter 9 of reference 65]. The term “aluminium salt” also refers to any of the “hydroxide” or “phosphate” adjuvants that are in general use as adjuvants. In some embodiments, which permit aluminium salts, the use of an aluminium hydroxide adjuvant is preferred.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 65]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. Mean particle diameters in the range of 1-10 μm are reported in reference 66. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [chapter 9 of ref. 65]. The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants. The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

A mixture of both an aluminium hydroxide and an aluminium phosphate has can also be used. In this situation there may be more aluminium phosphate than hydroxide e.g. a weight ratio of at least 2:1 e.g. ≧5:1, ≧6:1, ≧7:1, ≧8:1, ≧9:1, etc.

In some embodiments of the invention (e.g. wherein the immunogenic composition comprises a purified MenB LOS) the composition may comprise: (i) an aluminium hydroxide, an immunostimulatory oligonucleotide and a polycationic polymer; (ii) an aluminium phosphate, an immunostimulatory oligonucleotide and a polycationic polymer; or (iii) an aluminium hydroxide, an aluminium phosphate, an immunostimulatory oligonucleotide and a polycationic polymer.

The concentration of Al⁺⁺⁺ in a pharmaceutical composition of the invention will usually be <10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml.

### Adsorption

Preferred complexes of immunostimulatory oligonucleotide and polycationic polymer are adsorptive i.e. immunogens can adsorb to the complexes, by a variety of mechanisms. In some circumstances, however, immunogen and complex can both be present in a composition without adsorption, either through an intrinsic property of the immunogen or because of steps taken during formulation (e.g. the use of an appropriate pH during formulation to prevent adsorption from occurring).

Aluminium salt adjuvants are also adsorptive. In embodiments where a complex and an aluminium salt are both present, therefore, there can be multiple adsorptive opportunities for an immunogen: an immunogen can adsorb to aluminium salt, to a oligonucleotide/polymer complex, to both (in various proportions), or to neither. The invention covers all such arrangements. For example, in one embodiment an immunogen can be adsorbed to an aluminium salt, and the adsorbed immunogen/salt can then be mixed with an oligonucleotide/polymer complex. In another embodiment an immunogen can be adsorbed to an oligonucleotide/polymer complex, and the adsorbed immunogen/complex can then be mixed with an aluminium salt. In another embodiment two immunogens (the same or different) can be separately adsorbed to an oligonucleotide/polymer complex and to an aluminium salt, and the two adsorbed components can then be mixed.

In some situations, an immunogen may change its adsorption status e.g. by a change in pH or temperature, or after mixing of components. Desorption of antigens from aluminium salts in vitro [67] and in vivo [68] is known. Desorption from one adsorptive particle followed by resorption to a different adsorptive particle can occur, thereby resulting in e.g. transfer of an immunogen from an aluminium salt adjuvant to a complex or vice versa. In some embodiments, a single antigen molecule or complex might adsorb to both an aluminium salt and a complex, forming a bridge between the two adsorptive particles.

If an immunogen adsorbs to an adsorptive component, it is not necessary for all of the immunogen to adsorb. This situation can occur because of an immunogen's intrinsic equilibrium between adsorbed and soluble phases, or because adsorptive surfaces are saturated. Thus the immunogen in a composition may be fully or partially adsorbed, and the adsorbed fraction can be on one or more different adsorptive components (e.g. on aluminium salt and/or on a oligonucleotide/polymer complex). In this situation, the adsorbed fraction may be at least 10% (by weight) of the total amount of that immunogen in the composition e.g. >20%, >30%, >40%, >50%, >60%, >70%, >80%, >90%, >95%, >98% or more. In some embodiments an immunogen is totally adsorbed i.e. none is detectable in the supernatant after centrifugation to separate complexes from bulk liquid medium. In other embodiments, though, none of a particular immunogen may be adsorbed.

In some circumstances it is possible that the immunostimulatory oligonucleotide and/or polycationic polymer component of a complex could adsorb to an aluminium salt. Preferably, though, the complexes remain intact after mixing with an aluminium salt. Also, to avoid adsorption of complexes to an aluminium salt (and vice versa) it is useful that the aluminium salt and the complexes have similar points of zero charge (isoelectric points) e.g. within 1 pH unit of each other. Thus useful complexes have a PZC of between 10 and 12, which is useful for combining with an aluminium hydroxide adjuvant having a PZC of about 11.

### The Oil-in-Water Emulsion

Most embodiments do not contain an “oil-in-water” emulsion, although some embodiments permit their presence e.g. where the immunogenic composition comprises a purified MenB LOS Oil-in-water emulsions typically include at least one surfactant, with the oil(s) and surfactant(s) being biodegradable (metabolisable) and biocompatible.

The oil droplets in the emulsion are generally less than 5 nm in diameter, and ideally have a sub-micron diameter, with these small sizes being achieved with a microfluidiser to provide stable emulsions. Droplets with a size less than 220 nm are preferred as they can be subjected to filter sterilization. In some useful emulsions at least 80% (by number) of the oil droplets have a diameter less than 500 nm.

The emulsions can include oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil, etc. In the grain group, corn oil is the most readily available, but the oil of other cereal grains such as wheat, oats, rye, rice, teff, triticale, etc. may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, are readily available from commercial sources or may be obtained by methods known in the art. Squalene is preferred.

Other useful oils are the tocopherols, which are advantageously included in vaccines for use in elderly subjects (e.g. aged 60 years or older) because vitamin E has been reported to have a positive effect on the immune response in this subject group. They also have antioxidant properties that may help to stabilize emulsions. Various tocopherols exist (α, β, β, δ, ε or ξ) but α is usually used. A preferred α-tocopherol is DL-α-tocopherol. α-tocopherol succinate is known to be compatible with influenza vaccines and to be a useful preservative as an alternative to mercurial compounds.

Mixtures of oils can be used e.g. squalene and α-tocopherol.

An oil content in the range of 2-20% (by volume) is typical.

Surfactants can be classified by their ‘HLB’ (hydrophile/lipophile balance). Some surfactants useful with the invention have a HLB of at least 10 e.g. at least 15 or at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. The most preferred surfactant for including in the emulsion is polysorbate 80 (polyoxyethylene sorbitan monooleate; Tween 80).

Mixtures of surfactants can be used e.g. Tween 80/Span 85 mixtures. A combination of a polyoxyethylene sorbitan ester and an octoxynol is also suitable. Another useful combination comprises laureth 9 plus a polyoxyethylene sorbitan ester and/or an octoxynol.

Useful amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, e.g. 0.1 to 10% and in particular 0.1 to 1% or about 0.5%.

Squalene-containing emulsions are preferred, particularly those containing polysorbate 80.

Specific oil-in-water emulsion adjuvants useful with the invention include, but are not limited to:
A submicron emulsion of squalene, polysorbate 80, and sorbitan trioleate. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [69-71], as described in more detail in Chapter 10 of ref. 65 and chapter 12 of ref. 72. The MF59 emulsion advantageously includes citrate ions e.g. 10 mM sodium citrate buffer.
A submicron emulsion of squalene, a tocopherol, and polysorbate 80. These emulsions may have from 2 to 10% squalene, from 2 to 10% tocopherol and from 0.3 to 3% polysorbate 80, and the weight ratio of squalene:tocopherol is preferably ≦1 (e.g. 0.90) as this can provide a more stable emulsion. Squalene and polysorbate 80 may be present at a volume ratio of about 5:2 or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion has submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [73].
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL. The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [74] (0.05-1% Thr-MDP, 5% squalane, 2.5% Pluronic L121 and 0.2% polysorbate 80). It can also be used without the Thr-MDP, as in the “AF” adjuvant [75] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [76]. The emulsion may also include one or more of: alditol; a cryoprotective agent (e.g. a sugar, such as dodecylmaltoside and/or sucrose); and/or an alkylpolyglycoside. The emulsion may include a TLR4 agonist [77]. Such emulsions may be lyophilized.
An emulsion of squalene, poloxamer 105 and Abil-Care [78]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 79, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 80, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion in which a saponin (e.g. QuilA or QS21) and a sterol (e.g. a cholesterol) are associated as helical micelles [81].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [82].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [82].

As mentioned above, oil-in-water emulsions comprising squalene are particularly preferred. In some embodiments, the squalene concentration in a vaccine dose may be in the range of 5-15 mg (i.e. a concentration of 10-30 mg/ml, assuming a 0.5 ml dose volume). It is possible, though, to reduce the concentration of squalene [83,84] e.g. to include <5 mg per dose, or even <1.1 mg per dose. For example, a human dose may include 9.75 mg squalene per dose (as in the FLUAD™ product: 9.75 mg squalene, 1.175 mg polysorbate 80, 1.175 mg sorbitan trioleate, in a 0.5 ml dose volume), or it may include a fractional amount thereof e.g. ¾, ⅔, ½, ⅖, ⅓, ¼, ⅕, ⅙, 1/7, ⅛, 1/9, or 1/10. For example, a composition may include 4.875 squalene per dose (and thus 0.588 mg each of polysorbate 80 and sorbitan trioleate), 3.25 mg squalene/dose, 2.438 mg/dose, 1.95 mg/dose, 0.975 mg/dose, etc. Any of these fractional dilutions of the FLUAD™-strength MF59 can be used with the invention, while maintaining a squalene:polysorbate-80:sorbitan-trioleate ratio of 8.3:1:1 (by mass).

Further Antigens for Use with the Invention

Compositions and kits of the invention can also comprise one or more further antigens from other pathogens, particularly from bacteria and/or viruses. Preferred one or more further antigens are selected from:
a pneumococcal antigen
a diphtheria toxoid (‘D’)
a tetanus toxoid (‘T’)
a pertussis antigen (‘P’), which is typically acellular (‘aP’)
a hepatitis B virus (HBV) surface antigen (‘HBsAg’)
a hepatitis A virus (HAV) antigen
a conjugated Haemophilus influenzae type b capsular saccharide (‘Hib’)
inactivated poliovirus vaccine (IPV)
a conjugated N. meningitidis serogroup A capsular saccharide (‘MenA’)
a conjugated N. meningitidis serogroup W135 capsular saccharide (‘MenW135’)
a conjugated N. meningitidis serogroup Y capsular saccharide (‘MenY’)

One or more further antigen can be used. The following combinations of antigens are particularly preferred for use in compositions and kits of the invention:
MenC-PnC.
D-T-Pa-MenC.
D-T-Pa-Hib-MenC; D-T-Pa-IPV-MenC; D-T-Pa-HBsAg-MenC; D-T-Pa-MenC-PnC.
D-T-Pa-HBsAg-IPV-MenC; D-T-Pa-HBsAg-MenC-PnC.
D-T-Pa-HBsAg-IPV-Hib-MenC; D-T-Pa-HBsAg-Hib-MenC-MenA.
D-T-Pa-HBsAg-IPV-Hib-MenC-MenA; D-T-Pa-HBsAg-IPV-Hib-MenC-PnC.

These compositions may consist of the antigens listed, or may further include antigens from additional pathogens. Thus they can be used individually, or as components of further vaccines.

### Conjugated N. Meningitidis Saccharides

Further antigens can include conjugated meningococcal antigens. Conjugated meningococcal antigens comprise capsular saccharide antigens from Neisseria meningitidis conjugated to carrier proteins. Conjugated monovalent vaccines against serogroup C have been approved for human use, and include MENJUGATE™ [85], MENINGITECT™ and NEISVAC-C™. Mixtures of conjugates from serogroups A+C are known [86,87] and mixtures of conjugates from serogroups A+C+W135+Y have been reported [88-91] and were approved in 2005 as the MENACTRA™ product.

The invention may include saccharide from one or more of serogroups A, C, W135 and/or Y e.g. A, C, W135, Y, A+C, C+W135, C+Y, A+C+W135, A+C+Y, C+W135+Y, A+C+W135+Y.

The meningococcal serogroup A capsular saccharide is a homopolymer of (α1→6)-linked N-acetyl-D-mannosamine-1-phosphate, with partial O-acetylation in the C3 and C4 positions. Acetylation at the C-3 position can be 70-95%. Conditions used to purify the saccharide can result in de-O-acetylation (e.g. under basic conditions), but it is preferred to retain OAc at this C-3 position. Thus, preferably at least 50% (e.g. at least 60%, 70%, 80%, 90%, 95% or more) of the mannosamine residues are O-acetylated at the C-3 position.

The meningococcal serogroup C capsular saccharide is an α2→9-linked homopolymer of sialic acid (N-acetylneuraminic acid), typically with O-acetyl (OAc) groups at C-7 or C-8 residues. The compound is represented as: →9)-Neu p NAc 7/8 OAc-(α2→. Some MenC strains (˜12% of invasive isolates) produce a polysaccharide that lacks this OAc group. The presence or absence of OAc groups generates unique epitopes, and the specificity of antibody binding to the saccharide may affect its bactericidal activity against O-acetylated (OAc−) and de-O-acetylated (OAc+) strains [92-94]. Licensed MenC conjugate vaccines include both OAc− (NEISVAC-C™) and OAc+ (MENJUGATE™ & MENINGITECT™) saccharides. Serogroup C saccharides used with the invention may be prepared from either OAc+ or OAc− strains. Preferred strains for production of serogroup C conjugates are OAc+ strains, preferably of serotype 16, preferably of serosubtype P1.7a,1. Thus C:16:P1.7a,1 OAc+ strains are preferred. OAc+ strains in serosubtype P1.1 are also useful, such as the C11 strain.

The serogroup W135 saccharide is a polymer of sialic acid-galactose disaccharide units. Like the serogroup C saccharide, it has variable O-acetylation, but at sialic acid 7 and 9 positions [95]. The structure is written as: →4)-D-Neup5Ac(7/9OAc)-α-(2→6)-D-Gal-α-(1→

The serogroup Y saccharide is similar to the serogroup W135 saccharide, except that the disaccharide repeating unit includes glucose instead of galactose. Like serogroup W135, it has variable O-acetylation at sialic acid 7 and 9 positions [95]. The serogroup Y structure is written as:

→4)-D-Neup5Ac(7/9OAc)-α-(2→6)-D-Glc-α-(1→

The MENJUGATE™ and MENINGITECT™ products use a CRM197 carrier protein, and this carrier can also be used according to the invention. The NEISVAC-C™ product uses a tetanus toxoid carrier protein, and this carrier can also be used according to the invention, as can diphtheria toxoid. Another useful carrier protein for the meningococcal conjugates is protein D from Haemophilus influenzae, which is not present in any existing approved conjugate vaccines.

The saccharide of further antigens may comprise full-length saccharides as prepared from meningococci, and/or it may comprise fragments of full-length saccharides. The saccharides of further antigens are preferably shorter than the native capsular saccharides seen in bacteria. Thus the saccharides of further antigens are preferably depolymerised, with depolymerisation occurring after saccharide purification but before conjugation. Depolymerisation reduces the chain length of the saccharides. One depolymerisation method involves the use of hydrogen peroxide [88]. Hydrogen peroxide is added to a saccharide (e.g. to give a final H₂O₂ concentration of 1%), and the mixture is then incubated (e.g. at about 55° C.) until a desired chain length reduction has been achieved. Another depolymerisation method involves acid hydrolysis [89]. Other depolymerisation methods are known in the art. The saccharides used to prepare conjugates for use according to the invention may be obtainable by any of these depolymerisation methods. Depolymerisation can be used in order to provide an optimum chain length for immunogenicity and/or to reduce chain length for physical manageability of the saccharides. Preferred saccharides have the following range of average degrees of polymerisation (Dp): A=10-20; C=12-22; W135=15-25; Y=15-25. In terms of molecular weight, rather than Dp, preferred ranges are, for all serogroups: <100 kDa; 5 kDa-75 kDa; 7 kDa-50 kDa; 8 kDa-35 kDa; 12 kDa-25 kDa; 15 kDa-22 kDa.

Meningococcal conjugates with a saccharide:protein ratio (w/w) of between 1:10 (i.e. excess protein) and 10:1 (i.e. excess saccharide) may be used in further antigens e.g. ratios between 1:5 and 5:1, between 1:2.5 and 2.5:1, or between 1:1.25 and 1.25:1. A ratio of 1:1 can be used.

Typically, a composition will include between 1 μg and 20 μg (measured as saccharide) per dose of each further antigen serogroup that is present.

Meningococcal conjugates may or may not be adsorbed to an aluminium salt adjuvant.

Meningococcal conjugates may be lyophilised prior to use according to the invention. If lyophilised, the composition may include a stabiliser such as mannitol. It may also include sodium chloride.

### Conjugated Pneumococcal Saccharides

Further antigens can include conjugated pneumococcal antigens. Conjugated pneumococcal antigens comprise capsular saccharide antigens from Streptococcus pneumoniae conjugated to carrier proteins [e.g. refs. 96 to 98]. It is preferred to include saccharides from more than one serotype of S. pneumoniae: mixtures of polysaccharides from 23 different serotype are widely used, as are conjugate vaccines with polysaccharides from between 5 and 11 different serotypes [99]. For example, PREVNAR™ [100] contains antigens from seven serotypes (4, 6B, 9V, 14, 18C, 19F, and 23F) with each saccharide individually conjugated to CRM197 by reductive amination, with 2 μg of each saccharide per 0.5 ml dose (4 μg of serotype 6B).

Further antigens preferably include saccharide antigens for at least serotypes 6B, 14, 19F and 23F. Further serotypes are preferably selected from: 1, 3, 4, 5, 7F, 9V and 18C. 7-valent (as in PREVNAR™), 9-valent (e.g. the 7 serotypes from PREVNAR, plus 1 & 5), 10-valent (e.g. the 7 serotypes from PREVNAR, plus 1, 5 & 7F) and 11-valent (e.g. the 7 serotypes from PREVNAR, plus 1, 3, 5 & 7F) coverage of pneumococcal serotypes is particularly useful.

The saccharide moiety of the conjugate may comprise full-length saccharides as prepared from pneumococci, and/or it may comprise fragments of full-length saccharides. The saccharides used according to the invention are preferably shorter than the native capsular saccharides seen in bacteria, as described above for meningococcal conjugates.

Pneumococcal conjugates with a saccharide:protein ratio (w/w) of between 1:10 (i.e. excess protein) and 10:1 (i.e. excess saccharide) may be used e.g. ratios between 1:5 and 5:1, between 1:2.5 and 2.5:1, or between 1:1.25 and 1.25:1.

The PREVNAR™ product use a CRM197 carrier protein, and this carrier can also be used according to the invention. Alternative carriers for use with pneumococcal saccharides include, but are not limited to, a tetanus toxoid carrier, a diphtheria toxoid carrier, and/or a H. influenzae protein D carrier. The use of multiple carriers for mixed pneumococcal serotypes may be advantageous [101] e.g. to include both a H. influenzae protein D carrier and e.g. a tetanus toxoid carrier and/or a diphtheria toxoid carrier. For example, one or more (preferably all) of serotypes 1, 4, 5, 6B, 7F, 9V, 14 and 23F may be conjugated to a H. influenzae protein D carrier, serotype 18C may be conjugated to a tetanus toxoid, and serotype 19F may be conjugated to a diphtheria toxoid carrier.

Typically, a composition will include between 1 μg and 20 μg (measured as saccharide) per dose of each serotype that is present.

### Pertussis Antigens

Further antigens can include pertussis antigens. Bordetella pertussis causes whooping cough. Pertussis antigens in vaccines are either cellular (whole cell, in the form of inactivated B. pertussis cells) or acellular. Preparation of cellular pertussis antigens is well documented [e.g. see chapter 21 of ref. 102] e.g. it may be obtained by heat inactivation of phase I culture of B. pertussis. Preferably, however, the invention uses acellular antigens.

Where acellular antigens are used, it is preferred to use one, two or (preferably) three of the following antigens: (1) detoxified pertussis toxin (pertussis toxoid, or ‘PT’); (2) filamentous hemagglutinin (‘FHA’); (3) pertactin (also known as the ‘69 kiloDalton outer membrane protein’). These three antigens are preferably prepared by isolation from B. pertussis culture grown in modified Stainer-Scholte liquid medium. PT and FHA can be isolated from the fermentation broth (e.g. by adsorption on hydroxyapatite gel), whereas pertactin can be extracted from the cells by heat treatment and flocculation (e.g. using barium chloride). The antigens can be purified in successive chromatographic and/or precipitation steps. PT and FHA can be purified by, for example, hydrophobic chromatography, affinity chromatography and size exclusion chromatography. Pertactin can be purified by, for example, ion exchange chromatography, hydrophobic chromatography and size exclusion chromatography. FHA and pertactin may be treated with formaldehyde prior to use according to the invention. PT is preferably detoxified by treatment with formaldehyde and/or glutaraldehyde. As an alternative to this chemical detoxification procedure the PT may be a mutant PT in which enzymatic activity has been reduced by mutagenesis [103], but detoxification by chemical treatment is preferred.

Acellular pertussis antigens are preferably adsorbed onto one or more aluminium salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred.

Compositions will typically include: 1-50 μg/dose PT; 1-50 μg/dose FHA; and 1-50 μg pertactin. Preferred amounts are about 25 μg/dose PT, about 25 μg/dose FHA and about 8 μg/dose pertactin.

As well as PT, FHA and pertactin, it is possible to include fimbriae (e.g. agglutinogens 2 and 3) in an acellular pertussis vaccine.

### Inactivated Poliovirus Vaccine

Further antigens can include inactivated poliovirus antigens. Poliovirus causes poliomyelitis. Rather than use oral poliovirus vaccine, further antigens use IPV, as disclosed in more detail in chapter 24 of reference 102.

Polioviruses may be grown in cell culture, and a preferred culture uses a Vero cell line, derived from monkey kidney. Vero cells can conveniently be cultured on microcarriers. After growth, virions may be purified using techniques such as ultrafiltration, diafiltration, and chromatography. Prior to administration to patients, polioviruses must be inactivated, and this can be achieved by treatment with formaldehyde.

Poliomyelitis can be caused by one of three types of poliovirus. The three types are similar and cause identical symptoms, but they are antigenically very different and infection by one type does not protect against infection by others. It is therefore preferred to use three poliovirus antigens in the invention: poliovirus Type 1 (e.g. Mahoney strain), poliovirus Type 2 (e.g. MEF-1 strain), and poliovirus Type 3 (e.g. Saukett strain). The viruses are preferably grown, purified and inactivated individually, and are then combined to give a bulk trivalent mixture for use with the invention.

Quantities of IPV are typically expressed in the ‘DU’ unit (the “D-antigen unit” [104]). It is preferred to use between 1-100 DU per viral type per dose e.g. about 80 DU of Type 1 poliovirus, about 16 DU of type 2 poliovirus, and about 64 DU of type 3 poliovirus.

Poliovirus antigens are preferably not adsorbed to any aluminium salt adjuvant before being used to make compositions of the invention, but they may become adsorbed onto aluminum adjuvant(s) in the vaccine composition during storage.

### Diphtheria Toxoid

Further antigens can include diphtheria toxoid antigens. Corynebacterium diphtheriae causes diphtheria. Diphtheria toxin can be treated (e.g. using formalin or formaldehyde) to remove toxicity while retaining the ability to induce specific anti-toxin antibodies after injection. These diphtheria toxoids are used in diphtheria vaccines, and are disclosed in more detail in chapter 13 of reference 102. Preferred diphtheria toxoids are those prepared by formaldehyde treatment. The diphtheria toxoid can be obtained by growing C. diphtheriae in growth medium (e.g. Fenton medium, or Linggoud & Fenton medium), which may be supplemented with bovine extract, followed by formaldehyde treatment, ultrafiltration and precipitation. The toxoided material may then be treated by a process comprising sterile filtration and/or dialysis.

Quantities of diphtheria toxoid can be expressed in international units (IU). For example, the NIBSC supplies the ‘Diphtheria Toxoid Adsorbed Third International Standard 1999’ [105,106], which contains 160 IU per ampoule. As an alternative to the IU system, the ‘Lf’ unit (“flocculating units” or the “limes flocculating dose”) is defined as the amount of toxoid which, when mixed with one International Unit of antitoxin, produces an optimally flocculating mixture [107]. For example, the NIBSC supplies ‘Diphtheria Toxoid, Plain’ [108], which contains 300 LF per ampoule, and also supplies ‘The 1st International Reference Reagent For Diphtheria Toxoid For Flocculation Test’ which contains 900 LF per ampoule.

Compositions typically include between 20 and 80 Lf of diphtheria toxoid, typically about 50 Lf.

By IU measurements, compositions will typically include at least 30 IU/dose.

The diphtheria toxoid is preferably adsorbed onto an aluminium hydroxide adjuvant.

### Tetanus Toxoid

Further antigens can include tetanus toxoid antigens. Clostridium tetani causes tetanus. Tetanus toxin can be treated to give a protective toxoid. The toxoids are used in tetanus vaccines, and are disclosed in more detail in chapter 27 of reference 102. Preferred tetanus toxoids are those prepared by formaldehyde treatment. The tetanus toxoid can be obtained by growing C. tetani in growth medium (e.g. a Latham medium derived from bovine casein), followed by formaldehyde treatment, ultrafiltration and precipitation. The material may then be treated by a process comprising sterile filtration and/or dialysis.

Quantities of tetanus toxoid can be expressed in international units (IU). For example, the NIBSC supplies the ‘Tetanus Toxoid Adsorbed Third International Standard 2000’ [110,111], which contains 469 IU per ampoule. As an alternative to the IU system, the ‘Lf’ unit (“flocculating units” or the “limes flocculating dose”) is defined as the amount of toxoid which, when mixed with one International Unit of antitoxin, produces an optimally flocculating mixture [107]. For example, the NIBSC supplies ‘The 1st International Reference Reagent for Tetanus Toxoid For Flocculation Test’[112] which contains 1000 LF per ampoule.

Compositions will typically include between 5 and 50 Lf of diphtheria toxoid, typically about 20 Lf.

By IU measurements, compositions will typically include at least 40 IU/dose.

The tetanus toxoid may be adsorbed onto an aluminium hydroxide adjuvant, but this is not necessary (e.g. adsorption of between 0-10% of the total tetanus toxoid can be used).

### Hepatitis a Virus Antigens

Further antigens can include hepatitis A virus antigens. Hepatitis A virus (HAV) is one of the known agents which causes viral hepatitis. HAV vaccines are disclosed in chapter 15 of reference 102. A preferred HAV component is based on inactivated virus, and inactivation can be achieved by formalin treatment. Virus can be grown on human embryonic lung diploid fibroblasts, such as MRC-5 cells. A preferred HAV strain is HM175, although CR326F can also be used. The cells can be grown under conditions that permit viral growth. The cells are lysed, and the resulting suspension can be purified by ultrafiltration and gel permeation chromatography.

The amount of HAV antigen, measured in EU (Elisa Units), is typically at least about 500 EU/ml.

### Hepatitis B Virus Surface Antigen

Further antigens can include hepatitis B virus antigens. Hepatitis B virus (HBV) is one of the known agents which causes viral hepatitis. The HBV virion consists of an inner core surrounded by an outer protein coat or capsid, and the viral core contains the viral DNA genome. The major component of the capsid is a protein known as HBV surface antigen or, more commonly, ‘HBsAg’, which is typically a 226-amino acid polypeptide with a molecular weight of ˜24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg has been made in two ways. The first method involves purifying the antigen in particulate form from the plasma of chronic hepatitis B carriers, as large quantities of HBsAg are synthesized in the liver and released into the blood stream during an HBV infection. The second way involves expressing the protein by recombinant DNA methods. HBsAg for use with the method of the invention is preferably recombinantly expressed in yeast cells. Suitable yeasts include, for example, Saccharomyces (such as S. cerevisiae) or Hanensula (such as H. polymorpha) hosts.

The HBsAg is preferably non-glycosylated. Unlike native HBsAg (i.e. as in the plasma-purified product), yeast-expressed HBsAg is generally non-glycosylated, and this is the most preferred form of HBsAg for use with the invention, because it is highly immunogenic and can be prepared without the risk of blood product contamination.

The HBsAg will generally be in the form of substantially-spherical particles (average diameter of about 20 nm), including a lipid matrix comprising phospholipids. Yeast-expressed HBsAg particles may include phosphatidylinositol, which is not found in natural HBV virions. The particles may also include a non-toxic amount of LPS in order to stimulate the immune system [113]. Preferred HbsAg is in the form of particles including a lipid matrix comprising phospholipids, phosphatidylinositol and polysorbate 20.

All known HBV subtypes contain the common determinant ‘a’. Combined with other determinants and subdeterminants, nine subtypes have been identified: ayw1, ayw2, ayw3, ayw4, ayr, adw2, adw4, adrq− and adrq+. Besides these subtypes, other variants have emerged, such as HBV mutants that have been detected in immunised individuals (“escape mutants”). The most preferred HBV subtype for use with the invention is subtype adw2.

In addition to the ‘S’ sequence, a surface antigen may include all or part of a pre-S sequence, such as all or part of a pre-S1 and/or pre-S2 sequence.

A preferred method for HBsAg purification involves, after cell disruption: ultrafiltration; size exclusion chromatography; anion exchange chromatography; ultracentrifugation; desalting; and sterile filtration. Lysates may be precipitated after cell disruption (e.g. using a polyethylene glycol), leaving HBsAg in solution, ready for ultrafiltration.

After purification HBsAg may be subjected to dialysis (e.g. with cysteine), which can be used to remove any mercurial preservatives such as thimerosal that may have been used during HBsAg preparation [114].

Quantities of HBsAg are typically expressed in micrograms, and a typical amount of HBsAg per vaccine dose is between 5 and 5 μg e.g. 10 μg/dose.

Although HBsAg may be adsorbed to an aluminium hydroxide adjuvant in the final vaccine (as in the well-known ENGERIX-B™ product), or may remain unadsorbed, it will generally be adsorbed to an aluminium phosphate adjuvant [115].

Conjugated Haemophilus influenzae Type b Antigens

Further antigens can include conjugated Haemophilus influenzae type b (‘Hib’) antigens. Hib causes bacterial meningitis. Hib vaccines are typically based on the capsular saccharide antigen [e.g. chapter 14 of ref. 102], the preparation of which is well documented [e.g. references 116 to 125].

The Hib saccharide can be conjugated to a carrier protein in order to enhance its immunogenicity, especially in children. Typical carrier proteins are tetanus toxoid, diphtheria toxoid, the CRM197 derivative of diphtheria toxoid, H. influenzae protein D, and an outer membrane protein complex from serogroup B meningococcus. The carrier protein in the Hib conjugate is preferably different from the carrier protein(s) in the meningococcal conjugate(s), but the same carrier can be used in some embodiments.

Tetanus toxoid is the preferred carrier, as used in the product commonly referred to as ‘PRP-T’. PRP-T can be made by activating a Hib capsular polysaccharide using cyanogen bromide, coupling the activated saccharide to an adipic acid linker (such as (1-ethyl-3-(3-dimethylaminopropyl) carbodiimide), typically the hydrochloride salt), and then reacting the linker-saccharide entity with a tetanus toxoid carrier protein.

The saccharide moiety of the conjugate may comprise full-length polyribosylribitol phosphate (PRP) as prepared from Hib bacteria, and/or fragments of full-length PRP.

Hib conjugates with a saccharide:protein ratio (w/w) of between 1:5 (i.e. excess protein) and 5:1 (i.e. excess saccharide) may be used e.g. ratios between 1:2 and 5:1 and ratios between 1:1.25 and 1:2.5. In preferred vaccines, however, the weight ratio of saccharide to carrier protein is between 1:2 and 1:4, preferably between 1:2.5 and 1:3.5. In vaccines where tetanus toxoid is present both as an antigen and as a carrier protein then the weight ratio of saccharide to carrier protein in the conjugate may be between 1:0.3 and 1:2 [126].

Amounts of Hib conjugates are generally given in terms of mass of saccharide (i.e. the dose of the conjugate (carrier+saccharide) as a whole is higher than the stated dose) in order to avoid variation due to choice of carrier. A typical amount of Hib saccharide per dose is between 1-30 μg, preferably about 10 μg.

Administration of the Hib conjugate preferably results in an anti-PRP antibody concentration of ≧0.15 μg/ml, and more preferably ≧1 μg/ml, and these are the standard response thresholds.

Hib conjugates may be lyophilised prior to their use according to the invention. Further components may also be added prior to freeze-drying e.g. as stabilizers. Preferred stabilizers for inclusion are lactose, sucrose and mannitol, as well as mixtures thereof e.g. lactose/sucrose mixtures, sucrose/mannitol mixtures, etc. The final vaccine may thus contain lactose and/or sucrose. Using a sucrose/mannitol mixture can speed up the drying process.

Hib conjugates may or may not be adsorbed to an aluminium salt adjuvant. It is preferred not to adsorb them to an aluminium hydroxide adjuvant.

Mixing of Oligonucleotide and Polymer with MenB Antigen

Immunogenic compositions of the invention can conveniently be prepared by mixing an aqueous suspension of the oligonucleotide/polymer complex with an antigen. The complex is typically maintained in liquid form, hence providing an easy way of co-formulating them.

In some embodiments one or both of the suspensions includes an immunogen so that the mixing provides an immunogenic composition of the invention.

Where two liquids are mixed the volume ratio for mixing can vary (e.g. between 20:1 and 1:20, between 10:1 and 1:10, between 5:1 and 1:5, between 2:1 and 1:2, etc.) but is ideally about 1:1. The concentration of components in the two suspensions can be selected so that a desired final concentration is achieved after mixing e.g. both may be prepared at 2× strength such that 1:1 mixing provides the final desired concentrations.

Various concentrations of oligonucleotide and polycationic polymer can be used e.g. any of the concentrations used in references 58, 61, 62 or 127. For example, a polycationic oligopeptide can be present at 1100 μM, 1000 μM, 350 μM, 220 μM, 200 μM, 110 μM, 100 μM, 11 μM, 10 μM, 1 μM, 500 nM, 50 nM, etc. An oligonucleotide can be present at 44 nM, 40 nM, 20 nM, 14 nM, 4.4 nM, 4 nM, 2 nM, etc. A polycationic oligopeptide concentration of less than 2000 nM is typical. For SEQ ID NOs: 1 & 2, mixed at a molar ratio of 1:25, the concentrations in mg/mL in three embodiments of the invention may thus be 0.311 & 1.322, or 0.109 & 0.463, or 0.031 and 0.132.

Some immunogenic compositions of the invention comprise an aluminium salt and a complex of the immunostimulatory oligonucleotide and polycationic polymer. In such compositions, an aluminium salt and a complex of the immunostimulatory oligonucleotide and polycationic polymer are typically both particulate. The mean particle diameter of aluminium salt adjuvants is typically in the order of 1-20 μm [66,128]. This is also the size range for complexes seen in IC31™. When such particles are combined, the average diameter of the salt particles may be substantially the same as the average diameter of the complexes. In other embodiments, however, the average diameter of the salt particles may be smaller than the average size of the complexes. In other embodiments, the average diameter of the salt particles may be larger than the average size of the complexes. Where the average diameters differ, the larger diameter may be greater by a factor of at least 1.05× e.g. 1.1×, 1.2×, 1.3×, 1.4×, 1.5×, 2×, 2.5×, 3× or more. If either the salt or the complex has particles with a range of diameters, but the average diameters differ, the ranges may or may not overlap. Thus the largest salt particle may be smaller than the smallest complex particles, or the largest complex particles may be smaller than the smallest salt particles.

Because the particles are generally too large to be filter sterilised, sterility of an immunogenic composition of the invention will typically be achieved by preparing the complex, and where appropriate, the aluminium salt, under sterile conditions, and then mixing them under sterile conditions. For instance, the components of the complex could be filter sterilised. In some embodiments, these sterile complexes could then be mixed with an autoclaved (sterile) aluminium salt adjuvant to provide a sterile adjuvant composition. This sterile adjuvant can then be mixed with a sterile immunogen to give an immunogenic composition suitable for patient administration.

The density of aluminium salt particles is typically different from the density of a complex of immunostimulatory oligonucleotide and polycationic polymer, which means that the two particles might be separated based on density e.g. by sucrose gradient.

### Pharmaceutical Compositions

Immunogenic compositions of the invention usually include components in addition to the MenB antigen and the oligonucleotide and polymer e.g. they typically include one or more pharmaceutically acceptable component. Such components may also be present in immunogenic compositions of the invention, originating either in the adjuvant composition or in another composition. A thorough discussion of such components is available in reference 129.

A composition may include a preservative such as thiomersal or 2-phenoxyethanol. It is preferred that the vaccine should be substantially free from (e.g. <10 μg/ml) mercurial material e.g. thiomersal-free. Vaccines containing no mercury are more preferred. Preservative-free vaccines are particularly preferred. α-tocopherol succinate can be included as an alternative to mercurial compounds in influenza vaccines.

To control tonicity, a composition may include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate, and/or magnesium chloride, etc.

Compositions may have an osmolality of between 200 mOsm/kg and 400 mOsm/kg, e.g. between 240-360 mOsm/kg, maybe within the range of 280-330 mOsm/mg or 290-310 mOsm/kg.

The pH of a composition will generally be between 5.0 and 8.1, and more typically between 6.0 and 8.0 e.g. 6.5 and 7.5, or between 7.0 and 7.8.

A composition is preferably sterile. A composition is preferably non-pyrogenic e.g. containing <1 EU (endotoxin unit, a standard measure) per dose, and preferably <0.1 EU per dose. A composition is preferably gluten free.

An immunogenic composition may include material for a single immunisation, or may include material for multiple immunisations (i.e. a ‘multidose’ kit). The inclusion of a preservative is useful in multidose arrangements. As an alternative (or in addition) to including a preservative in multidose compositions, the compositions may be contained in a container having an aseptic adaptor for removal of material.

Compositions will generally be in aqueous form at the point of administration. Vaccines are typically administered in a dosage volume of about 0.5 ml, although a half dose (i.e. about 0.25 ml) may sometimes be administered e.g. to children. In some embodiments of the invention a composition may be administered in a higher dose e.g. about 1 ml e.g. after mixing two 0.5 ml volumes.

### Packaging of Compositions or Kit Components

Suitable containers for immunogenic compositions and kit components of the invention include vials, syringes (e.g. disposable syringes), etc. These containers should be sterile. The containers can be packaged together to form a kit e.g. in the same box.

Where a component is located in a vial, the vial can be made of a glass or plastic material. The vial is preferably sterilized before the composition is added to it. To avoid problems with latex-sensitive subjects, vials are preferably sealed with a latex-free stopper, and the absence of latex in all packaging material is preferred. The vial may include a single dose of vaccine, or it may include more than one dose (a ‘multidose’ vial) e.g. 10 doses. Useful vials are made of colorless glass. Borosilicate glasses are preferred to soda lime glasses. Vials may have stoppers made of butyl rubber.

A vial can have a cap (e.g. a Luer lock) adapted such that a syringe can be inserted into the cap. A vial cap may be located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached, a separate needle may be supplied with the syringe for assembly and use. Such a needle may be sheathed. The plunger in a syringe may have a stopper to prevent the plunger from being accidentally removed during aspiration. The syringe may have a latex rubber cap and/or plunger. Disposable syringes contain a single dose of vaccine. The syringe will generally have a tip cap to seal the tip prior to attachment of a needle, and the tip cap may be made of a butyl rubber. If the syringe and needle are packaged separately then the needle is preferably fitted with a butyl rubber shield. Useful syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

It is usual in multi-component products to include more material than is needed for subject administration, so that a full final dose volume is obtained despite any inefficiency in material transfer. Thus an individual container may include overfill e.g. of 5-20% by volume.

### Methods of Treatment, and Administration of Immunogenic Compositions

Compositions of the invention are suitable for administration to human subjects, and the invention provides a method of raising an immune response in a subject, comprising the step of administering an immunogenic composition of the invention to the subject.

The invention also provides a method of raising an immune response in a subject, comprising the step of mixing the contents of the containers of a kit of the invention and administering the mixed contents to the subject.

The invention also provides composition or kit of the invention for use as a medicament e.g. for use in raising an immune response in a subject.

The invention also provides the use of a MenB antigen (as defined above), an immunostimulatory oligonucleotide and a polycationic polymer, in the manufacture of a medicament for raising an immune response in a subject.

These methods and uses will generally be used to generate an antibody response, preferably a protective antibody response.

Immunogenic compositions of the invention can be administered in various ways. The usual immunisation route is by intramuscular injection (e.g. into the arm or leg), but other available routes include subcutaneous injection, intranasal, oral, buccal, sublingual, intradermal, transcutaneous, transdermal, etc.

Immunogenic compositions prepared according to the invention may be used as vaccines to treat both children and adults. A subject may be less than 1 year old, 1-5 years old, 5-15 years old, 15-55 years old, or at least 55 years old. Subjects for receiving the vaccines may be elderly (e.g. ≧50 years old, ≧60 years old, and preferably ≧65 years), the young (e.g. ≦5 years old), hospitalised subjects, healthcare workers, armed service and military personnel, pregnant women, the chronically ill, immunodeficient subjects, people travelling abroad, etc. Aluminium salt adjuvants are routinely used in infant populations, and IC31™ has also been effective in this age group [127,130]. The vaccines are not suitable solely for these groups, however, and may be used more generally in a population.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve subjects. Multiple doses will typically be administered at least 1 week apart (e.g. about 2 weeks, about 3 weeks, about 4 weeks, about 6 weeks, about 8 weeks, about 12 weeks, about 16 weeks, etc.).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, or for viral growth, it is preferably one that has been approved for use in human vaccine production e.g. as in Ph Eur general chapter 5.2.3.

### MODES FOR CARRYING OUT THE INVENTION

### Adjuvants

IC31 complexes were prepared as disclosed in reference 62. An aluminium hydroxide adjuvant suspension is prepared by standard methods. Where compositions comprise an aluminium hydroxide adjuvant and IC31, adjuvant combinations were made by mixing the aluminium hydroxide adjuvant with IC31 complexes.

For Meningococcus (iii) and (iv) below, IC31 was prepared in high and low concentrations (10-fold difference) as disclosed in reference 62 and a squalene-in-water emulsion. For Meningococcus (iv), MF59, was prepared as disclosed in Chapter 10 of reference 65. Adjuvant combinations were made by mixing MF59 with IC31^{high} or IC31^{low} at either a 1:1 volume ratio or a 5:1 volume ratio.

### Meningococcus (i)

The three polypeptides which make up the ‘5CVMB’ vaccine disclosed in reference 1 were adjuvanted with aluminium hydroxide and/or IC31. The polypeptides have amino acid sequences SEQ ID NO: 13, SEQ ID NO: 14 and SEQ ID NO: 15 (see refs. 1 and 131)

In a first set of experiments, nine groups of mice received 10 μg of antigens, 3 mg/ml of aluminium hydroxide and varying doses of IC31. Groups received the following nine compositions, with groups 7-9 receiving the same antigens as 1-6 but differently formulated:

| | Antigen dose (μg) | IC31 volume* (μl) | Al—H (mg/ml) |
|---|---|---|---|
| 1 | 10 | 100 | 3 |
| 2 | 10 | 50 | 3 |
| 3 | 10 | 25 | 3 |
| 4 | 10 | 10 | 3 |
| 5 | 10 | 0 | 3 |
| 6** | 10 | 100 | 0 |
| 7 | 10 | 0 | 3 |
| 8 | 10 | 100 | 3 |
| 9** | 10 | 100 | 0 |

A standard IC31 suspension was used. 100 μl of this suspension gave full-strength. Lower volumes gave lower strengths. To preserve the volume for the lower-strength compositions, buffer was added up to 100 μl.
**Embodiments of the invention.

Sera from the mice were tested against a panel of meningococcal strains for bactericidal activity. Bactericidal titers from experiment MP03 were as follows against six different strains, A to F:

| | A | B | C | D | E | F |
|---|---|---|---|---|---|---|
| 1 | >65536 | 4096 | 8192 | 4096 | 256 | 32768 |
| 2 | >65536 | 8192 | 8192 | 8192 | 512 | >65536 |
| 3 | >65536 | 4096 | 4096 | 8192 | 512 | 32768 |
| 4 | >65536 | 2048 | 4096 | 4096 | 512 | 8192 |
| 5 | >65536 | 2048 | 4096 | 8192 | 256 | 32768 |
| 6 | >65536 | 4096 | >8192 | 8192 | 1024 | >65536 |
| 7 | >65536 | 2048 | 4096 | 4096 | 256 | 4096 |
| 8 | >65536 | >8192 | >8192 | >8192 | 512 | >65536 |
| 9 | 32768 | 8192 | >8192 | >8192 | 4096 | >65536 |

Thus the titers obtained with Al—H as the only adjuvant (group 5) were generally improved across the panel by the addition of IC31 at various ratios (groups 1 to 4). The same effect was seen with the different antigen formulation (compare groups 7 and 8).

Moreover, when IC31 was used as the only adjuvant, (groups 6 and 9), bactericidal titers were found to be as high, or higher, than Al—H and IC31+Al—H, in all six strains.

The nine compositions were tested for pH and osmolality. For compositions 1-5, 7 and 8 the pH was in the range of 6.2 to 6.6; compositions 6 and 9 had a slightly higher pH, in the range 6.9 to 7.3. Osmolality of all compositions was in the range of 280-330 mOsm/kg.

### Meningococcus

A triple-fusion polypeptide containing three variants of fHBP, in the order II-III-I (as disclosed in reference 60; SEQ ID NO: 17 herein), was adjuvanted with aluminium hydroxide and/or IC31.

In a first set of experiments, six groups of mice received 20 μg of antigen (with or without a purification tag), 3 mg/ml of aluminium hydroxide and 100 μl of IC31. Groups received the following:

| | Antigen dose | | | |
|---|---|---|---|---|
| | (μg) | Antigen tag | IC31 volume (μl) | Al—H (mg/ml) |
| 1** | 20 | No | 100 | 0 |
| 2** | 20 | Yes | 100 | 0 |
| 3 | 20 | No | 100 | 3 |
| 4 | 20 | Yes | 100 | 3 |
| 5 | 20 | No | 0 | 3 |
| 6 | 20 | Yes | 0 | 3 |

**Embodiments of the invention.

Sera from the mice were tested against a panel of meningococcal strains for bactericidal activity.

Sera from experiment MP05 were again tested against a panel of strains (25 in total). 56% of strains in group 1 (IC31, no tag) and group 3 (IC31+Al—H, no tag) had a titer≧1:1024, while only 36% of strains in group 5 (Al—OH, no tag) had a titer≧1:1024. Similarly, 76% of strains in groups 1 and 3 had a titer≧1:128 while this titer was only observed in 64% of strains in group 5. Thus, in the absence of a purification tag, the highest bactericidal titers were achieved using IC31.

Bactericidal titer comparisons of purification-tagged antigens revealed that 84% of strains in group 2 (IC31, tag) had a titer of ≧1:128. By contrast, 80% of strains in group 4 (IC31+Al—H) and only and 76% of strains in group 6 (Al—OH) had a titer of ≧1:128. Thus, in the presence of a purification tag, highest bacterial titers were achieved with IC31 alone.

The tag-free compositions (1, 3 and 5) were tested for pH and osmolality. The pH was in the range of 6.87 to 7.00. Osmolality was in the range of 302-308 mOsm/kg.

Further immunogenicity experiments used the fHBP_{II-III-I} antigen in combination with the NadA and 287-953 antigens (SEQ ID NOs: 13 and 15) in experiment MP04, with the same groupings and strain panel. Groups 1 and 3 had a bactericidal titer of ≧1:128 in 100% of strains tested, compared to only 84% in group 5. With a more stringent threshold of ≧1:1024, sera from groups 1 and 3 were bactericidal against 88% of strains, compared to only 56% in group 5.

Similar results were observed with purification-tagged antigens, where 88% of groups 2 and 4 had a bactericidal titer of ≧1:128 compared to only 80% of group 6.

Thus, the highest anti-meningococcus immune responses were obtained with IC31 alone, which was at least as good as IC31+Al—H and better than Al—H alone.

Meningococcus (iii)

The three polypeptides which make up the ‘5CVMB’ vaccine disclosed in reference 1 were combined with a tetravalent mixture of meningococcal conjugates against serogroups A, C, W135 and Y. The mixture was adjuvanted with Al—H and/or IC31 (at high or low concentration). Bactericidal titers were as follows against a panel with one strain from each of serogroups A, C, W135 and Y:

| | A | C | W135 | Y |
|---|---|---|---|---|
| Un-immunised | <16 | <16 | <16 | <16 |
| No adjuvant | 1024 | 256 | 128 | 512 |
| IC31^{high}** | 32768 | 16384 | 4096 | 4096 |
| IC31^{low}** | 16384 | 8192 | 1024 | 2048 |
| Al-hydroxide | 16384 | 8192 | 1024 | 4096 |
| Al—H + IC31^{high} | 16384 | 32768 | 4096 | 8192 |
| Al—H + IC31^{low} | 8192 | 65536 | 2048 | 8192 |

**Embodiments of the invention.

Thus the best titers against serogroup A were seen when using IC31 alone, and titers against serogroups C, W135 and Y were higher than when using Al—H alone.

### Meningococcus (iv)

The antigens from the meningococcus serogroup B vaccine of reference 1 were adjuvanted with MF59, IC31^{high}, IC31^{low} or combinations thereof. Sera from immunised mice were tested for their bactericidal activity against various meningococcal strains. Representative results include:

| Strain→ | A | B | C | D | E | F | G | H |
|---|---|---|---|---|---|---|---|---|
| IC31^{low}** | 1024 | 256 | 4096 | 2048 | 256 | 64 | 512 | <16 |
| MF59 + IC31^{low} | 4096 | 1024 | 4096 | 2048 | 1024 | 128 | 4096 | <16 |
| MF59 | 32768 | 1024 | 32768 | 4096 | 2048 | 128 | 4096 | <16 |
| MF59 + IC31^{high} | 8192 | 2048 | 8192 | 32768 | 2048 | 128 | 8192 | <16 |
| IC31^{high}** | 16384 | 2048 | 16384 | 32768 | 2048 | 512 | 4096 | <16 |

**Embodiments of the invention.

Use of IC31 alone elicited the highest bactericidal titers in strains B, D, E, and F, and the second highest titers in strains A, C, and G.

These meningococcal B protein antigens were also combined with conjugated saccharide antigens from serogroups A, C, W135 and Y antigens and were tested with the same adjuvant mixtures. Bactericidal titers against a test strain from each serogroup were as follows:

| Antigen→ | A | C | W135 | Y |
|---|---|---|---|---|
| IC31^{low}** | 16384 | 8192 | 1024 | 2048 |
| MF59 + IC31^{low} | 4096 | 8192 | 4096 | 8192 |
| MF59 | 16384 | 8192 | 2048 | 4096 |
| MF59 + IC31^{high} | 8192 | 16384 | 4096 | 4096 |
| IC31^{high}** | 32768 | 16384 | 4096 | 4096 |

**Embodiments of the invention.

Therefore, the highest bactericidal titers were seen when using IC31 for serogroup A, C and W135.

### Meningococcus (v)

A composition containing the three variants of fHBP, in the order II-III-I, +961+287-953 (denoted rMenB1) was adjuvanted with Al—H, IC31, or IC31+Al—H. These compositions were compared with a composition comprising 936−741+961+287−953+OMV, which was adjuvanted with Al—H (rMenB2).

Sera from immunised mice were tested for their bactericidal activity against 12 meningococcal strains. rMenB1 adjuvanted with IC31 alone was found to elicit a higher % coverage across the 12 strains tested than any other composition (e.g. with >90% coverage, compared to 50% coverage for rMenB2).

It will be understood that the invention has been described by way of example only and modifications may be made whilst remaining within the scope and spirit of the invention.

### REFERENCES

[1] Giuliani et al. (2006) Proc Natl Acad Sci USA 103(29):10834-9.
[2] Tettelin et al. (2000) Science 287:1809-1815.
[3] WO00/66741.
[4] WO99/57280
[5] Martin et al. (1997) J Exp Med 185(7):1173-83.
[6] WO96/29412.
[7] Perkins-Balding et al. (2003) Microbiology 149:3423-35.
[8] WO01/55182.
[9] WO01/38350.
[10] WO00/23595.
[11] Madico et al. (2006) J. Immunol. 177:501-10.
[12] Schneider et al. (2006) J. Immunol. 176:7566-75.
[13] Ram et al. (2003) J Biol Chem 278:50853-62.
[14] WO98/53851
[15] U.S. Pat. No. 6,531,131
[16] WO00/26384.
[17] U.S. Pat. No. 6,645,503
[18] WO03/070282.
[19] WO94/08021
[20] WO2004/015099.
[21] WO2007/144316.
[22] WO2007/144317.
[23] WO02/09643.
[24] Katial et al. (2002) Infect. Immun. 70:702-707.
[25] U.S. Pat. No. 6,180,111.
[26] WO01/34642.
[27] WO2004/019977.
[28] EP-0011243.
[29] Fredriksen et al. (1991) NIPH Ann. 14(2):67-80.
[30] WO01/91788.
[31] WO2005/004908.
[32] Maiden et al. (1998) PNAS USA 95:3140-3145.
[33] WO98/56901.
[34] WO2006/081259.
[35] Claassen et al. (1996) 14(10):1001-8.
[36] WO99/10497.
[37] Steeghs et al. (2001) The EMBO Journal 20:6937-6945.
[38] WO01/52885.
[39] WO00/25811.
[40] WO01/09350.
[41] WO02/09746.
[42] WO02/062378.
[43] WO2004/014417.
[44] WO03/105890.
[45] WO2006/024946
[46] Krieg (2003) Nature Medicine 9:831-835.
[47] McCluskie et al. (2002) FEMS Immunology and Medical Microbiology 32:179-185.
[48] WO98/40100.
[49] U.S. Pat. No. 6,207,646.
[50] U.S. Pat. No. 6,239,116.
[51] U.S. Pat. No. 6,429,199.
[52] WO01/93905.
[53] WO02/32451.
[54] Aichinger et al. (2008) Cell Biology International 32:1449-58.
[55] Alvarez-Bravo et al. (1994) Biochem J302:535-8.
[56] Nakajima et al. (1997) FEBS Letts 415:64-66.
[57] Fritz et al. (2004) Vaccine 22:3274-84.
[58] Schellack et al. (2006) Vaccine 24:5461-72.
[59] Lingnau et al. (2007) Expert Rev Vaccines 6:741-6.
[60] WO2004/084938.
[61] Kamath et al. (2008) Eur J Immunol 38:1247-56.
[62] Riedl et al. (2008) Vaccine 26:3461-8.
[63] Kritsch et al. (2005) J Chromatography B 822:263-70.
[64] Lingnau et al. (2003) Vaccine 20:3498-508.
[65] Vaccine Design: The Subunit and Adjuvant Approach (Powell & Newman) 1995 (ISBN 0-306-44867-X).
[66] Clausi et al. (2008) J Pharm Sci DOI 10.1002/jps.21390
[67] WO2009/081172.
[68] Seeber et al. (1991) J Parenteral Sci Technol 45:156-9.
[69] WO90/14837.
[70] Podda & Del Giudice (2003) Expert Rev Vaccines 2:197-203.
[71] Podda (2001) Vaccine 19: 2673-2680.
[72] Vaccine Adjuvants: Preparation Methods and Research Protocols (Volume 42 of Methods in Molecular Medicine series). ISBN: 1-59259-083-7. Ed. O'Hagan.
[73] WO2008/043774.
[74] Allison & Byars (1992) Res Immunol 143:519-25.
[75] Hariharan et al. (1995) Cancer Res 55:3486-9.
[76] US-2007/0014805.
[77] US-2007/0191314.
[78] Suli et al. (2004) Vaccine 22(25-26):3464-9.
[79] WO95/11700.
[80] U.S. Pat. No. 6,080,725.
[81] WO2005/097181.
[82] WO2006/113373.
[83] WO2007/052155.
[84] WO2008/128939.
[85] Jones (2001) Curr Opin Investig Drugs 2:47-49.
[86] Costantino et al. (1992) Vaccine 10:691-8.
[87] Lieberman et al. (1996) JAMA 275:1499-503.
[88] WO02/058737.
[89] WO03/007985.
[90] Rennels et al. (2002) Pediatr Infect Dis J21:978-979.
[91] Campbell et al. (2002) J Infect Dis 186:1848-1851.
[92] Arakere & Frasch (1991) Infect. Immun. 59:4349-4356.
[93] Michon et al. (2000) Dev. Biol. 103:151-160.
[94] Rubinstein & Stein (1998) J. Immunol. 141:4357-4362.
[95] WO2005/033148.
[96] Watson (2000) Pediatr Infect Dis J19:331-332.
[97] Rubin (2000) Pediatr Clin North Am 47:269-285, v.
[98] Jedrzejas (2001) Microbiol Mol Biol Rev 65:187-207.
[99] Zielen et al. (2000) Infect. Immun. 68:1435-1440.
[100] Darkes & Plosker (2002) Paediatr Drugs 4:609-630.
[101] WO98/51339.
[102] Vaccines. (eds. Plotkin & Orenstein). 4th edition, 2004, ISBN: 0-7216-9688-0.
[103] Rappuoli et al. (1991) TIBTECH 9:232-238.
[104] Module 6 of WHO's The immunological basis for immunization series (Robertson)
[105] Sesardic et al. (2001) Biologicals 29:107-22.
[106] NIBSC code: 98/560.
[107] Module 1 of WHO's The immunological basis for immunization series (Galazka).
[108] NIBSC code: 69/017.
[109] NIBSC code: DIFT.
[110] Sesardic et al. (2002) Biologicals 30:49-68.
[111] NIBSC code: 98/552.
[112] NIBSC code: TEFT.
[113] Vanlandschoot et al. (2005) J Gen Virol 86:323-31.
[114] WO03/066094.
[115] WO93/24148.
[116] Ramsay et al. (2001) Lancet 357(9251):195-196.
[117] Lindberg (1999) Vaccine 17 Suppl 2:S28-36.
[118] Buttery & Moxon (2000) J R Coll Physicians Lond 34:163-168.
[119] Ahmad & Chapnick (1999) Infect Dis Clin North Am 13:113-133, vii.
[120] Goldblatt (1998) J. Med. Microbiol. 47:563-567.
[121] European patent 0477508.
[122] U.S. Pat. No. 5,306,492.
[123] WO98/42721.
[124] Conjugate Vaccines (eds. Cruse et al.) ISBN 3805549326, particularly vol. 10:48-114.
[125] Hermanson (1996) Bioconjugate Techniques ISBN: 0123423368 or 012342335X.
[126] WO96/40242.
[127] Olafsdottir et al. (2009) Scand J Immunol 69:194-202.
[128] Hem & HogenEsch (2007) Expert Rev Vaccines 6:685-98.
[129] Gennaro (2000) Remington: The Science and Practice of Pharmacy. 20th edition, ISBN: 0683306472.
[130] Kamath et al. (2008) PLoS ONE 3(11):e3683.
[131] WO2004/032958.

1. An immunogenic composition comprising (i) a meningococcal serogroup B antigen and (ii) an adjuvant comprising an immunostimulatory oligonucleotide and a polycationic polymer; wherein (i) the immunogenic composition does not include an aluminium salt; (ii) the immunogenic composition does not include an oil-in-water emulsion; (iii) the meningococcal serogroup B antigen does not include a polypeptide comprising an amino acid sequence selected from SEQ ID NOs 13, 14, 15, 16, 17, 18, 19, 20, 21 or 22; and (iv) the immunogenic composition does not include a fHBP antigen.

2. An immunogenic composition comprising (i) a meningococcal serogroup B antigen; (ii) an adjuvant comprising an immunostimulatory oligonucleotide and a polycationic polymer and; (iii) one or more further antigens selected from a pneumococcal antigen, a diphtheria toxoid, tetanus toxoid, a pertussis antigen, HBsAg, a HAV antigen, a Hib antigen, and/or IPV.

3. An immunogenic composition comprising (i) a purified meningococcal lipooligosaccharide; and (ii) an adjuvant comprising an immunostimulatory oligonucleotide and a polycationic polymer.

4. The immunogenic composition of claim 2, wherein said immunogenic composition further comprises one or more of (i) an aluminium salt; and (ii) an oil-in-water emulsion.

5. The immunogenic composition of claim 1 wherein the oligonucleotide and the polymer are associated with each other to form a complex.

6. The immunogenic composition of claim 1, wherein the immunostimulatory oligonucleotide is single-stranded and has between 10 and 100 nucleotides.

7. The immunogenic composition of claim 6, wherein the oligonucleotide is 5′-(IC)₁₃-3′.

8. The immunogenic composition of claim 1, wherein the polycationic polymer is a peptide.

9. The immunogenic composition of claim 8, wherein the peptide includes one or more Leu-Leu dipeptide sequence(s), one or more Lys-Lys dipeptide sequence(s), and/or one or more Arg-Arg dipeptide sequence(s).

10. The immunogenic composition of claim 8, wherein the peptide includes one or more Lys-Leu dipeptide sequence(s) and/or one or more Lys-Leu-Lys tripeptide sequence(s).

11. The immunogenic composition of claim 8, wherein the peptide has between 5 and 50 amino acids.

12. The immunogenic composition of claim 11, wherein the peptide has amino acid sequence KLKLLLLLKLK.

13. The immunogenic composition of claim 1, wherein the oligonucleotide and polymer are present at a molar ratio 1:25.

14. A process for preparing the immunogenic composition of claim 1, comprising a step of mixing (i) an immunostimulatory oligonucleotide and a polycationic polymer and (ii) a meningococcal serogroup B antigen.

15. A kit comprising: (i) a first container that contains an immunostimulatory oligonucleotide and a polycationic polymer and (ii) a second container that contains a meningococcal serogroup B antigen; wherein the immunogenic composition does not include an aluminium salt; (ii) the immunogenic composition does not include an oil-in-water emulsion; (iii) the meningococcal serogroup B antigen does not include peptide with SEQ IDs 13, 14, 15, 16, 17, 18, 19, 20, 21 or 22; and (iv) the immunogenic composition does not include a fHBP antigen.

16. A kit comprising (i) a first container that contains an immunostimulatory oligonucleotide and a polycationic polymer and (ii) a second container that contains a meningococcal serogroup B antigen wherein said meningococcal serogroup B antigen is a purified meningococcal lipooligosaccharide.

17. A kit comprising which comprises (i) a container that contains an immunostimulatory oligonucleotide and a polycationic polymer and (ii) a container that contains a meningococcal serogroup B antigen and (iii) a container that contains one or more further antigens selected from pneumococcal saccharide antigen, diphtheria toxoid, tetanus toxoid, pertussis antigen, HBsAg, HAV antigen, Hib antigen, and/or IPV.

18. An immunogenic composition comprising (i) a 5-valent antigen component consisting of a MenB antigen, a conjugated capsular saccharide from serogroup A N. meningitidis, a conjugated capsular saccharide from serogroup C N. meningitidis, a conjugated capsular saccharide from serogroup W135 N. meningitidis, a conjugated capsular saccharide from serogroup Y N. meningitidis; and (ii) an adjuvant comprising an immunostimulatory oligonucleotide and a polycationic polymer, provided that the immunogenic composition does not include an aluminium salt and does not include an oil-in-water emulsion.
